(19) United States
(12) Patent Application Publication
(10) Pub. No.: US 2015/0023892 A1
(43) Pub. Date: Jan. 22, 2015

(54) MOUTH WASH COMPOSITIONS FOR DENTURE ADHESIVE REMOVAL

(71) Applicant: Nian Wu, North Brunswick, NJ (US)

(72) Inventor: Nian Wu, North Brunswick, NJ (US)

(21) Appl. No.: 14/332,692

(22) Filed: Jul. 16, 2014

Related U.S. Application Data
(60) Provisional application No. 61/846,731, filed on Jul. 16, 2013.

(51) Int. Cl. A61K 8/86; A61K 8/60; A61K 8/06; A61K 8/97; A61Q 11/02; A61Q 11/00; A61K 8/34

(52) U.S. Cl. CPC A61K 8/86; A61Q 11/00; A61K 8/608; A61K 8/34; A61K 8/345; A61K 8/97; A61Q 11/02; A61K 8/06; A61K 2800/74; A61K 2800/10
USPC 424/58; 424/49; 510/116

(57) ABSTRACT

An antiseptic, polymer-lipid or emulsification agents-based aqueous composition can be used for intraoral removal of denture adhesive residuals. More particularly, the present invention relates to employing a polymer-lipid based or a mixture of polymer-lipids and emulsification agents or a mixture of emulsification agents and Co-emulsification agents for convenient and effective removing the denture adhesive residuals intra mouth or a combination of two ore more polymer-lipids or emulsification agents or Co-emulsification agents thereof.

This application claims priority to the provisional patent application serial number: 61/846,731, entitled “Mouth Wash Compositions for Denture Adhesive Removal”, filed in the U.S. Patent and Trademark Office on Jul. 16, 2013, by Nian Wu, which is hereby incorporated by reference.

### FIELD OF THE INVENTION

This invention relates to methods and compositions for removing of denture adhesive residuals associated with antiseptic integrants for mouth wash. More particularly, the present invention relates to employing polymer-lipid conjugates, such as polyethylene glycol based amphophilic lipid conjugates and other emulsification agents or polymer-lipid based surfactants or Co-emulsification agent(s), such as polyethylene glycol, propylene glycols, anionic surfactants or cationic surfactants or fatty alcohols for formulating oral compositions with antiseptic integrants having increased emulsification or removability of conversional denture adhesives.

### BACKGROUND OF THE INVENTION

The main ingredients of denture adhesives include carbonyl methyl cellulose (CMC), vegetable gums, xantham acacia, polyethylene oxide polymers, cationic polyacryl amide polymers and polyvinyl pyrrolidone (povidone). Long acting and less soluble of monoalkyl esters of poly (methyl vinyl ether/maleic acid) in series of copolymers with varying ester groups (PVM/MA salts), which display molecular cross linking may also be used.

Typically, dentures within the mouth may be secured by using the cream prepared from finely ground particles of the gums instead of the powder forms. While many efforts have been made over the years to develop better denture adhesive compositions with improved dental care features, only few products are available for intramouth adhesive removal.

It may be difficult to remove the denture adhesive material from the soft gum tissue and palate inside of the month after the denture devices are removed. There are several solution or solvent based inventions published for the removal of denture adhesives, for example, U.S. Pat. Nos. 4,701, 223 and 4,807, 649 (gel and spraying liquid denture cleansers comprising a water soluble detergent based on sulfonated, sulfate and sulfoacetate fatty alcohols), U.S. Pat. No. 6,518, 227 (an oil-based solvent composition comprising tea tree oil in vegetable oils), US Patent Application 20,070,037,717 (Denture adhesive solvent compositions and disclosing an oil-based composition including sodium lauryl sulfate). Other physical devices such as brushes and wet wipes (U.S. Pat. Nos. 5,032, 082; 5,261, 817 and 5,987, 689) have been used to remove denture adhesive by physically wiping and scrubbing that may cause discomfort to the users. Therefore effective formulation for cleaning denture adhesive residuals from the denture and the oral cavity and, is still remaining as a challenge, what is needed are more effective methods for denture adhesive removal.

### SUMMARY OF THE INVENTION

Emulsion dispersion can be used as a method for blending multiple polymers in the denture adhesive homogeneously with the disclosed oral rinse formulas; as a result, it cleans the adhesive residuals after removing the denture device. Unlike oil based compositions, a polymer-lipid based aqueous composition reduces interfacial tension and retards particle flocculation during emulsifying a denture adhesive with the oral rinse formulas following further rinsing off with water that may produce a smooth and moisture feeling on oral gums and mucosa.

Currently commercial denture adhesive products are majorly as cream pastes. The new generations of denture adhesive materials may be using various polymers, mineral oil and other wax-like ingredients in cream formulas. Using proper polymer mixtures provides a better cohesive strength, reducing dry mouth (xerostomia) and other discomfort effects, a combination of polyethylene oxide and hydroxypropylmethylcellulose polymers have become very popular adhesive vehicle for a denture adhesive formulation that may improve overall oral care quality. While these synthetic products offer a better performance than the gum based alternatives, they may be very difficult to remove from the dentures and oral mucosa with only water rinse.

In one aspect of the present invention, an aqueous formulation for the removal of oil or polymer-based denture adhesives is disclosed. Solutions having ingredients commonly used in preparing pharmaceutical formulations such as propylene glycol, glycerin and polyethelene glycol, Nonoxynol 9, Octoxynol, various surfactants such as Benzalkonium chloride, Sodium lauryl sulfate, Cetylpyridinium chloride, Tyloxapol and various PEG-lipid conjugates preferable with middle or long alkyl chains of 8 to 20 carbons such as Poloxamers (Poloxamer 124, 188, 237, 338 and 407), Polyoxyl 35 castor oil, Polyoxyl 40 hydrogenated castor oil, Polyoxyl 10 oleyl ether, Polyoxyl 20 cetylstearyl ether, Polyoxyl 40 stearate, Polysorbate 20, Polysorbate 40, Polysorbate 60, Polysorbate 80 and d-a-Tocopheryl-polyethylene glycol 1000 succinatehave (polyethylene glycol 1000-Vitamin E or Vitamin E TPGS) have been tested as the delivery vehicles for removing denture adhesives. It has been discovered that significant improvement in cleaning the adhesive residuals may be achieved in a combination of these vehicles.

In at least one aspect of the present disclosure, an oral composition for ingredients is provided. The composition comprises: a) an aqueous solution or mixture; b) a flavor; c) a preservative; d) a colorant; and e) an emulsification enhancer as the remover comprising polymers or polymer-lipids or emulsification agents.

In at least one other aspect of the present disclosure, an oral composition for mouth washing is provided. The composition comprises: i) an aqueous solution or mixture of antiseptic integrants; and ii) a removal enhancer comprising at least one polymer-lipid or emulsification agent.

In at least one aspect of the present disclosure, a process for making an oral composition for denture adhesive removal is provided. The process comprises the steps of: adding a polymer-lipid or emulsification agents to the vessel; mixing until all ingredients are visually dispersed in the solution; adding pre-dissolved excipients in water to the vessel; and mixing until a homogenous solution is achieved.

### BRIEF DESCRIPTION OF THE INVENTION

The invention comprises various polymer-lipids or emulsification agents based formulations of water soluble polymers and agents including compositions for oral preparations of denture adhesive removal. In one aspect, the invention comprises a solution of polymer(s) or polymer-lipid(s) or emulsification agent(s) to enhance the removability of denture adhesives in aqueous solutions.

In at least one aspect of the present disclosure, an oral composition for cleaning the denture adhesive residuals is provided. The composition comprises: an aqueous solution or mixture; a removal enhancer comprising at least one polymer or polymer-lipid or emulsification agent or Co-emulsification agent.

### DETAILED DESCRIPTION

Embodiments of the present invention are described herein in the context of emulsification enhancers for increasing the clearance or removal of denture adhesives. Those of ordinary skill in the art will realize that the following detailed description of the present invention is illustrative only and is not intended to be in any way limiting. Other embodiments of the present invention will readily suggest themselves to such skilled persons having the benefit of this disclosure. Reference will now be made in detail to implementations of the present invention as illustrated in the accompanying tables and examples.

In the interest of clarity, not all of the routine features of the implementations described herein are shown and described. It will, of course, be appreciated that in the development of any such actual implementation, numerous implementation-specific decisions may need be made in order to achieve specific goals, such as compliance with application and business related constraints, and that these specific goals may vary from one implementation to another and from one developer to another. However, development and implementation of the disclosed may be made for those of ordinary skill in the art having the benefit of this disclosure.

U.S. patent application Ser. No. 13/364,967 and 13/354,726, which are hereby incorporated by reference, may teach the formation of poor-water soluble pharmaceutical agents by employing certain lipid-carbohydrate-polyethleneglycol (LCP) conjugates. They may describe how to prepare the PEG-carbohydrate-lipid conjugates and its applications by simply adding the conjugate to an aqueous solution. It has been demonstrated that LCPs may be useful for removing denture adhesives with a simple formulation where less organic solvents or Co-emulsification agents will be required.

Polyethylenglycol (PEG) is widely used as a water soluble carrier for pharmaceutical applications. PEG may undoubtedly be the most studied and applied synthetic polymer in the biomedical field [Duncan, R. Nature Rev. Drug Discov. 2003, 2, 347-360]. As an uncharged, water-soluble, nontoxic, nonimmunogenic polymer, PEG may be an ideal material for biomedical applications. PEG possesses several beneficial properties: very low toxicity [Pang, S. N. J., J. Am. Coil. Toxicol, 12 (1993), 429-456], excellent solubility in aqueous solutions [Powell, G.M., Handbook of Water Soluble Gums and Resins, R.L.Davidson (Ed.), Ch. 18 (1980), MGraw-Hill, New York], and extremely low immunogenicity and antigenicity [Dreborg, S, Crit. Rev. Ther. Drug Carrier Syst., 6 (1990), 315-365]. The polymer is known to be non-biodegradable, yet it is readily excretable after administration into living organisms. In vitro study showed that its presence in aqueous solutions has shown no deleterious effect on protein conformation or activities of enzymes.

Propylene glycol is a mixture of di-, tri-, tetra- and polypropylene glycols. Propylene glycol is classified by the U. S. Food and Drug Administration as GRAS (generally recognized as safe) material for use as a direct food additive. Propylene glycol is an excellent dispersing agent for oils and polymers, commonly used as an emulsification agent in pharmaceutical dosage forms.

When used as an adhesive remover, a polymer-lipid conjugates or emulsification agent may have the capacity to improve the dispersibility of a polymer based adhesive mixture. Any liquid form GRAS polymer-lipid conjugates may be suitable as the vehicle. Selected polymer-lipid conjugates include but not limited to polyethylene glycol mono- or di-ester of fatty acids: PEG (400) dilaurate (MAPEG®), caprylocaproyl polyoxyl-8 glycerides (Labrasol®), polyoxyethylene mono- and di-ether of fatty acids (Brij® surfactants) or poloxamers (Pluracrae®, Pluronics®, Triton®), polyethoxylated castor oil (Cremophor or Kolliphor®), polyethylene glycol 1000-vitamin E (Kolliphor™ TPGS), polysorbates such as polyoxyethylene (n=20 to 80) sorbitan monolaurate (Alkest® TW or Tween), homo- and copolymers of acrylic acid crosslinked with a polyalkenyl polyether (Carbopol®), polycarbophil (Noveon®), ceteareth-n (polyoxyethylene ethers of a mixture of saturated fatty alcohols).

One aspects of the present disclosure, when used as an adhesive remover, an emulsification agent may have the capacity to improve the dispersibility of a polymer based adhesive mixtures. Aqueous oral solutions according to the present invention comprise Co-emulsification agents including and not limited to glycerol, t-octylphenoxypolyethoxyethanol, diethylene glycol monoethyl ether (Transcutol®), propylene glycol, polyethylene glycols, methoxyl polyethylene glycol ethers, triglycerides at concentrations ranging from 1 to 40 percent (w/v), wherein the concentration of polymer-lipid in the oral solution ranges from 0% to 20%.

At least one aspect of the present disclosure may provide aqueous solutions in which the remover consists of 0 to 40 percent (w/v) of a Co-emulsification agent or Co-emulsification agents, 1 to 30 percent (w/v) of emulsification agent(s) and 40 to 80 percent (v/v) of water or a buffer solution. Also preferable are aqueous oral solutions of this invention in which 50 to 70 percent (v/v) of the total solution is water or a buffer solution.

A first step for preparation of the oral solution may comprise combining an amphipathic PEG-lipid conjugate(s) or other emulsification agents, sugar alcohols which may be semisolid or solid at the temperature of solubilization, and a Co-emulsification agent(s). For formulating an oral solution at room temperature (which may be preferred), a concentrated solution of an emulsification agent(s) may be desired. Such preparation may be done by first adding the solid form of the emulsification agent(s) to the Co-emulsification agent(s). The concentrated solution may be further diluted with water or a buffered solution of sodium phosphate or sodium citrate with a pH between about 3 to about 7. Any solid additive(s) may be pre-dissolved in appropriate amounts of water, then mix with the PEG-lipids or other emulsification agents in aqueous solution.

The LCP lipids shown in Table 1 may be suitable for use in various aspects of the present disclosure. LCPs with oxy or amide or succinyl linkers (X=oxygen or carbonyl or succinyl) may be preferred, though LCPs with other linkers may be used.

Backbone (B) may comprise glycerol or glycerol-like analogues or linear amines (tri- or tetra-amines) or amino acids having three available binding sites; where the lipid may comprise carboxylic acids including and not limited to diacylglycerol or fatty acids or bile acids; sugar may comprise a carbohydrate including monosaccharides or disaccharides or oligosaccharides; X₁, X₂ and X₃ are the same or different linkers and X represents an oxy or single or replicate linkers or combination of two or more molecules in between the backbone and one of functional groups. The General Structure is meant to include all racemers or structural isomers of the structure, as they may be functionally equivalent. The PEG chain may be a single PEG or a branched PEG chains consisting of 5 to 45 subunits. There may be a terminal group (R) on the PEG chain which may comprise a wide variety of chemical moieties. In at least one aspect of the present disclosure, R has a molecular weight of less than about 650. The Lipid-carbohydrate-PEG conjugates may be useful for applications as a denture adhesive remover.

If a terminal group is attached to the PEG chain in Table 1, it may comprise a wide variety of chemical moieties. Such moieties may have a molecular weight of less than 650. Such moieties include —NH₂, —COOH, —OCH₂CH₃, —OCH₂CH₂OH, —COCH═CH₂, —OCH₂CH₂NH₂, —OSO₂CH₃, —OCH₂C₆H₆, —OCH₂COCH₂CH₂COONC₄H₄O₂, —CH₂CH₂═CH₂, C₁₀H₁₆N₂O₃S and —OC₆H₆. The terminal group may be a functional group that facilitates linking of therapeutic or targeting agents to the surface of lipid vesicle aggregates. Amino acids, amino alkyl esters, biotins, maleimide, diglycidyl ether, maleinimido propionate, methylcarbamate, tosylhydrazone salts, azide, propargyl-amine, propargyl alcohol, NHS esters (e.g., propargyl NHS ester, NHS-biotin, sulfo-NHS-LC-biotin, or NHS carbonate), hydrazide, succinimidyl ester, succinimidyl tartrate, succinimidyl succinate, and toluenesulfonate salt may be useful for such linking. Linked therapeutic and targeting agents may include Fab fragments (fragment antigen-binding), cell surface binding agents, and the like. Additionally, the terminal group may include functional cell-targeting ligands such as folate, transferrin and molecules such as monoclonal antibodies, ligands for cellular receptors or specific peptide sequences may be attached to the liposomal surface to provide specific binding sites. The terminal group may be neutral or include either negatively or positively charged head-groups such as decanolamine, octadecylolamine, octanolamine, butanolamine, dodecanolamine, hexanolamine, tetradecanolamine, hexadecanolamine, oleylamine, decanoltrimethylaminium, octadecyloltrimethylaminium, octanoltrimethyl-aminium, butanoltrimethylaminium, dodecanoltrimethylaminium, hexanoltrimethylaminium, tetradecanoltrimethylaminium, hexadecanoltrimethylaminium, oleyltrimethylaminium, for example. Other useful R groups may include alkyl groups such as alkoxy moieties, amino acids, and sugars including monosaccharides, disaccharides, trisaccharides and the oligosaccharides—containing 1, 2, 3, and 4 or more monosaccharide units respectively. Additionally, targeting moieties such as antibody fragments and vitamins may also be used as R groups. Generally, the R group may be highly soluble in water. The molecular weight of the R group may be less than about 650, and for most applications the R group may be easily polarized, in order to increase the binding and interaction with the main components of adhesives.

TABLE 1
LCP (Lipid-carbohydrate-polyethyleneglycols) may be used in the present invention

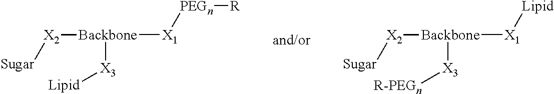

X₁, X₂ and X₃ represent linkers which may be oxy or thiol or carbonyl, amino or succinyl or
the like which may not be distinguished in the following name and detailed in the preceding
sections. X₁, X₂ and X₃ may be the same or different. The number of subunits in the PEG
polymer ranges from 6 to 16.

| Shorthand name | Name |
|---|---|
| MAGC-PEGs | monoacylglycerol-carbohydrate-polyethylene glycols |
| MAPC-PEGs | monoacylpolyamine-carbohydrate-polyethylene glycols |
| MAAC-PEGs | monoacylamino acid-carbohydrate-polyethylene glycol |
| ODL-TrpPEGs | oleoyldiethylenetriamine-tryptophanyl PEG |
| LOS-PEGs | N-lactobionyloleoyl-mPEG serinate |
| LOS-bioPEGs | N-lactobionyloleoyl-biotinylated PEG serinate |
| OAL-mPEG | oleoyl-N-(3-aminopropyl)propane-1,3-diamine- |
| | monomethoxypolyethylene glycol ether Lactobionate |
| OPL-mPEG | oleoylpropanediamine-monomethoxypolyethylene glycol ether |
| | Lactobionate |
| OAPDL-11 | oleoyl-N-(3-aminopropyl)propane-1,3-diamine-Undecaethylene |
| | glycol methyl ether Lactobionate |
| GDODL-12: | dioleoylglyceroldiethylenetriamine-monomethoxyl dodecaethylene |
| | glycol ether lactobionate |
| GMODL-12 | dimyristoylglycerol diethylenetriamine-monomethoxyl |
| | dodecaethylene glycol ether lactobionate |
| GML-12 | myristoylglycerol-dodecaethylene glycol lactobionate |
| GOL-12 | oleoylglycerol-dodecaethylene glycol lactobionate |
| MDTL-12 | myristoyldiethylenetetramine-dodecaethylene glycol lactobionate |
| ODL-12 | oleoyldiethylenetriamine-dodecaethylene glycol lactobionate |
| ODL-15 | oleoyldiethylenetriamine-pentadecaehylene glycol lactobionate |
| ODTL-12 | oleoyldiethylenetetramine-dodecaethylene glycol lactobionate |
| ODTL-15 | oleoyldiethylenetetramine-pentadecaethylene glycol lactobionate |
| MTL12 | myristoyltriethylenetetramine-dodecaethylene glycol lactobionate |
| OTL-12 | oleoyltriethylenetetramine-dodecaethylene glycol lactobionate |
| OTL-15 | oleoyltriethylenetetramine-pentadecaethylene glycol lactobionate |
| GDODL-12 | dioleoylglycerol-diethylenetriamine-monomethoxyl polyethylene |
| | glycol ether lactobionate |
| OAPEL-PEG | oleoyl(aminopropylamino)ethanoyl-mPEG Lactobionate |
| LOS-bioPEG | N-lactobionyloleoyl-biotinylated PEG Serinate |
| LOL-bioPEG | N-lactobionyloleoyl-biotinylated PEG Lycinate |
| DCAL-PEG | N-desoxycholylaspartate-mPEG lactobionate |
| OAL-mPEG | oleoylaminopropanediol-mPEG lactobionate |
| OAL-bioPEG | oleoylaminopropanediol-biotinylated PEG lactobionate |
| ODL-ThrPEG | oleoyldiethylenetriamine-threoninyl PEG lactobionate |
| ODL-bioPEG | oleoyldiethylenetriamine-biotinylated PEG lactobionate |
| ODL-PEG | oleoyldiethylenetriamine-PEG lactobionate |

The manufacture of the oral solution may comprise first adding an emulsification agent(s) to a Co-emulsification agent(s) and mixing until homogenous, which may be accomplished at room temperatures. Next, premixed aqueous integrants may be added to the mixture and mixed until a homogenous solution is obtained. Appropriate volumes of the solution may be filled into bottles using aseptic technique. While the formulated product may be stable at room temperature, it may be preferably stored under refrigeration for extended shelf life.

A preservative may be desired, the possible preservatives may be selected from a group of antimicrobial agents consisting of benzyl alcohol, chlorobutanol, methylparaben, propylparaben, phenol, ethylenediaminetetraacetic acid (EDTA), m-cresol, sorbic acid and its salts, benzoic acid and its salts, calcium propionate, sodium nitrite, sulfur dioxide, sodium bisulfite, and potassium hydrogen sulfite. The content of antimicrobial agents may preferably be between about 0.1 and 3 percent (w/v) of the total solution

In one aspect of the present disclosure, an oral solution composition for removing denture adhesive by rinsing is provided. The composition comprises an aqueous solution; an emulsification agent(s) or combination of emulsification agents and Co-emulsification agent(s); and a sugar alcohol as sweetener such mannitol or fucitol or sorbitol or xylitol at a concentration between about 0.5% and about 10%. The weight ratio of the emulsification agent to the Co-emulsification agents may be between about 0.01 and 10. The average MW of PEG chains in the PEG or mPEG may be less than about 1500. The content of water or a buffer may preferably be between about 40 and 80 percent (v/v) of the total solution.

Natural or artificial flavor may be added to improve a sensation of smell or mask the taste. The suitable artificial flavor may be selected from Peppermint (oil), Dare #13174 (Cherry), Menthol (Mint), Butanedione (Buttery), Isopentyl acetate (Banana), Benzaldehyde (Bitter almond), 3-Phenylprop-2-enal (Cinnamon), Ethyl propionate (Fruity), Methyl anthranilate (Grape), Limonene (Orange), Ethyl 2-trans-4-cis-decadienoate (Pear), Allyl hexanoate (Pineapple), Ethyl maltol (Sugar, Cotton candy), Ethylvanillin (Vanilla), and Methyl salicylate Wintergreen). The content of flavor may preferably be between about 0.1 and 2 percent (w/v) of the total solution.

The oral formulations may further comprise a color additive which may be selected from natural dyes including and not limited to Turmeric (E100), Cochineal (E120), Chlorophyllin (E140), Caramel coloring (E150), Saffron (E160a), Paprika (E160c), Annatto (E160b), Lycopene (E160d), Betanin (E162), Pandanus amaryllifolius, Clitoria ternatea or an artificial colorants approved by a government agency (EU or FDA) including and not limited to FD&C Blue No. 1, FD&C Blue No. 2, FD&C Green No. 3, FD&C Red No. 40, FD&C Red No. 3, FD&C Yellow No. 5, FD&C Yellow No. 6, Orange B, and Citrus Red 2. Finally an antimicrobial agent may be selected form a group of compounds consisting of benzyl alcohol, chlorobutanol, methylparaben, propylparaben, phenol, ethylenediaminetetraacetic acid (EDTA), m-cresol, sorbic acid and its salts, benzoic acid and its salts, calcium propionate, sodium nitrite, sulfur dioxide, sodium bisulfite, and potassium hydrogen sulfite. The content of colorant may preferably be between about 0.1 and 2 percent (w/v) of the total solution.

A general formula of oral solution for denture adhesive removal is represented in the following table (Table 2).

TABLE 2
Representative formulation of denture adhesive removal

| Ingredient | % |
|---|---|
| Cremophor (polyethoxylated castor oil) | 10 |
| Propylene Glycol | 5 |
| Polyethylene Glycol | 20 |
| Peppermint | 1.0 |
| Sodium citrate | 0.2 |
| Citric acid | 0.5 |
| Glycerol | 2.0 |
| Sorbitol | 10 |
| Benzoic acid | 0.5 |
| Salicylic acid | 0.01 |
| Saccharin sodium | 0.5 |
| Chlorhexidine digluconate | 0.2 |
| Green Tea Extra (1% tannin equivalence) | 1.0 |
| FD&C Green No. 3 | 0.1 |
| Purified Water | qs |

A general procedure for using the oral rinse solution is given hereby: measure a proper amount of (undiluted) rinse solution recommended (-30 mL) from the container or by a dentist. Close lips and keeping the teeth slightly apart, swish the liquid around in the mouth. Make sure to swish vigorously and thoroughly so that the rinse reaches the front and sides of the mouth equally. Being careful not to swallow, gargle by raising chin and saying “AAHH ” while holding the rinse in the mouth. This is especially helpful for reaching the back of the tongue, where adhesive may accumulate. Continue rinsing for about 30 seconds, and then completely spit the rinse from mouth. Repeat the above steps one more time as necessary and rinse with water after using the oral rinse solution.

The following examples intend to further illustrate the practice of the present invention.

### EXAMPLE 1

### Preparation of Oral Solution for Denture Adhesive Removal (E01)

PEG-lipid(s) was added to a vessel equipped with a mixer propeller. The Co-emulsification agent(s) was added with constant mixing. Pre-dissolved excipients in water were slowly added to the vessel with adequate mixing. Mixing continued until a homogenous solution was achieved. A sample formulation is described in Table 3.

TABLE 3

| Ingredient | % |
|---|---|
| Polysorbate 20 | 20 |
| Polyethylene glycol 600 | 10 |
| Polyethylene glycol 300 | 10 |
| Xylitol | 10 |
| Benzyl alcohol | 2.0 |
| Peppermint | 0.5 |
| FD&C Green No. 3 | 0.1 |
| Purified Water | qs 100 |

Polymer-lipid(s) may be selected from polyethylene glycol mono- or di-ester of fatty acids, polyoxyethylene mono- and di-ether of fatty acids or poloxamers, polyethoxylated castor oil, polyethylene glycol 1000-vitamin E, polysorbates such as polyoxyethylene sorbitan monolaurate, homo- and copolymers of acrylic acid crosslinked with a polyalkenyl polyether, polyoxyethylene ethers of a mixture of saturated fatty alcohols, and lipid-carbohydrate-PEG, where PEG chain contains 8 to 16 subunits. Emulsification agents 2 and 3 may be selected from glycerol, t-octylphenoxypolyethoxyethanol, diethylene glycol monoethyl ether, propylene glycol, polyethylene glycols, methoxyl polyethylene glycol ethers, benzalkonium chloride, sodium lauryl sulfate, nonoxynol-9, octoxynol, cetylpyridinium chloride, and triglycerides. Sweetener may select from a sugar alcohol such mannitol or fucitol or sorbitol or xylitol. The suitable artificial flavor may be selected from peppermint, cherry, cherry, menthol, mebutanedione, isopentyl acetate; benzaldehyde; 3-phenylprop-2-enal; ethyl propionate; methyl anthranilate; limonene; ethyl 2-trans-4-cis-decadienoate, allyl hexanoate; ethyl maltol; ethylvanillin; methyl salicylate. The antimicrobial agent(s) may be consisting of benzyl alcohol, chlorobutanol, methylparaben, propylparaben, phenol, ethylenediaminetetraacetic acid (EDTA), m-cresol, sorbic acid and its salts, benzoic acid and its salts, calcium propionate, sodium nitrite, sulfur dioxide, sodium bisulfite, potassium hydrogen sulfite. A colorant may be selected from turmeric, cochineal, chlorophyllin, caramel, saffron, paprika, annatto, lycopene, betanin, pandanus amaryllifolius, clitoria ternatea or an artificial colorant of FD&C Blue No. 1, FD&C Blue No. 2, FD&C Green No. 3, FD&C Red No. 40, FD&C Red No. 3, FD&C Yellow No. 5, FD&C Yellow No. 6, Orange B, and Citrus Red 2.

### EXAMPLE 2

### Preparation of Oral Solution for Denture Adhesive Removal (E02)

An oral rinsing solution suitable for denture adhesive removal was prepared the same as in Example 1. A sample formulation is described in Table 4.

TABLE 4

| Ingredient | % |
|---|---|
| Polysorbate 20 | 15 |
| Polyethylene glycol 600 | 15 |
| Polyethylene glycol 300 | 15 |
| Xylitol | 5 |
| benzyl alcohol | 2 |
| Peppermint | 1 |
| FD&C Green No. 3 | 0.1 |
| Purified Water | qs 100 |

Polymer-lipid(s) may be selected from polyethylene glycol mono- or di-ester of fatty acids, polyoxyethylene mono- and di-ether of fatty acids or poloxamers, polyethoxylated castor oil, polyethylene glycol 1000-vitamin E, polysorbates such as polyoxyethylene sorbitan monolaurate, homo- and copolymers of acrylic acid crosslinked with a polyalkenyl polyether, polyoxyethylene ethers of a mixture of saturated fatty alcohols, and lipid-carbohydrate-PEG, where PEG chain contains 8 to 16 subunits. Emulsification agents may be selected from Emulsification agents may be selected from glycerol, t-octylphenoxypolyethoxyethanol, diethylene glycol monoethyl ether, propylene glycol, polyethylene glycols, methoxyl polyethylene glycol ethers, benzalkonium chloride, sodium lauryl sulfite, notioxynol-9, octoxynol. cetylpyridinium chloride, and triglycerides. Sweetener may select from a sugar alcohol such mannitol or fucitol or sorbitol or xylitol. The suitable artificial flavor may be selected from peppermint, cherry, menthol, mebutanedione, isopentyl acetate; benzaldehyde; 3-phenylprop-2-enal; ethyl propionate; methyl anthranilate; limonene; ethyl 2-trans-4-cis-decadienoate, allyl hexanoate; ethyl maltol; ethylvanillin; methyl salicylate. The antimicrobial agent(s) may be consisting of benzyl alcohol, chlorobutanol, methylparaben, propylparaben, phenol, ethylenediaminetetraacetic acid (EDTA), m-cresol, sorbic acid and its salts, benzoic acid and its salts, calcium propionate, sodium nitrite, sulfur dioxide, sodium bisulfite, and potassium hydrogen sulfite. A colorant may be selected from turmeric, cochineal, chlorophyllin, caramel, saffron, paprika, annatto, lycopene, betanin, pandanus amaryllifolius, clitoria ternatea or an artificial colorant of FD&C Blue No. 1, FD&C Blue No. 2, FD&C Green No. 3, FD&C Red No. 40, FD&C Red No. 3, FD&C Yellow No. 5, FD&C Yellow No. 6, Orange B, and Citrus Red 2.

### EXAMPLE 3

### Preparation of Oral Solution for Denture Adhesive Removal (E03)

An oral rinsing solution suitable for denture adhesive removal was prepared the same as in Example 1. A sample formulation is described in Table 5.

TABLE 5

| Ingredient | % |
|---|---|
| Polysorbate 20 | 10 |
| Polyethylene glycol 600 | 25 |
| Polyethylene glycol 300 | 5 |
| benzyl alcohol | 2 |
| Peppermint | 0.5 |
| FD&C Green No. 3 | 0.1 |
| Purified Water | qs 100 |

Polymer-lipid(s) may be selected from polyethylene glycol mono- or di-ester of fatty acids, polyoxyethylene mono- and di-ether of fatty acids or poloxamers, polyethoxylated castor oil, polyethylene glycol 1000-vitamin E, polysorbates such as polyoxyethylene sorbitan monolaurate, homo- and copolymers of acrylic acid crosslinked with a polyalkenyl polyether, polyoxyethylene ethers of a mixture of saturated fatty alcohols and lipid-carbohydrate-PEG, where PEG chain contains 8 to 16 subunits. Emulsification agents may be selected from glycerol, t-octylphenoxypolyethoxyethanol, diethylene glycol monoethyl ether, propylene glycol, polyethylene glycols, methoxyl polyethylene glycol ethers, benzalkonium chloride, sodium lauryl sulfate, nonoxynol-9, octoxynol. cetylpyridinium chloride and triglycerides. Sweetener may select from a sugar alcohol such mannitol or fucitol or sorbitol or xylitol. The suitable artificial flavor may be selected from peppermint , cherry, menthol, mebutanedione, isopentyl acetate; benzaldehyde; 3-phenylprop-2-enal; ethyl propionate; methyl anthranilate; limonene; ethyl 2-trans-4-cis-decadienoate, allyl hexanoate; ethyl maltol; ethylvanillin; methyl salicylate. The antimicrobial agent(s) may be consisting of benzyl alcohol, chlorobutanol, methylparaben, propylparaben, phenol, ethylenediaminetetraacetic acid (EDTA), m-cresol, sorbic acid and its salts, benzoic acid and its salts, calcium propionate, sodium nitrite, sulfur dioxide, sodium bisulfite, and potassium hydrogen sulfite. A colorant may be selected from turmeric, cochineal, chlorophyllin, caramel, saffron, paprika, annatto, lycopene, betanin, pandanus amaryllifolius, clitoria ternatea or an artificial colorant of FD&C Blue No. 1, FD&C Blue No. 2, FD&C Green No. 3, FD&C Red No. 40, FD&C Red No. 3, FD&C Yellow No. 5, FD&C Yellow No. 6, Orange B, and Citrus Red 2.

### EXAMPLE 4

### Preparation of Oral Solution for Denture Adhesive Removal (E04)

An oral rinsing solution suitable for denture adhesive removal was prepared the same as in Example 1. A sample formulation is described in Table 6.

TABLE 6

| Ingredient | % (w/w)¹ |
|---|---|
| Polysorbate 20 | 5 |
| Polyethylene glycol 600 | 15 |
| Polyethylene glycol 300 | 20 |
| Sweetener | 5 |
| Benzyl alcohol | 2 |
| Peppermint | 0.5 |
| FD&C Green No. 3 | 0.1 |
| Purified Water | qs 100 |

¹w/w = weight to weight

Polymer-lipid may be selected from polyethylene glycol mono- or di-ester of fatty acids, polyoxyethylene mono- and di-ether of fatty acids or poloxamers, polyethoxylated castor oil, polyethylene glycol 1000-vitamin E, polysorbates such as polyoxyethylene sorbitan monolaurate, homo- and copolymers of acrylic acid crosslinked with a polyalkenyl polyether, polyoxyethylene ethers of a mixture of saturated fatty alcohols, and lipid-carbohydrate-PEG, where PEG chain contains 8 to 16 subunits. Emulsification agents may be selected from glycerol, t-octylphenoxypolyethoxyethanol, diethylene glycol monoethyl ether, propylene glycol, polyethylene glycols, methoxyl polyethylene glycol ethers, benzalkonium chloride, sodium lauryi sulfate, nonoxynol-9, octoxynol. cetylpyridinium chloride, and triglycerides. Sweetener may select from a sugar alcohol such mannitol or fucitol or sorbitol or xylitol. The suitable artificial flavor may be selected from peppermint, cherry, menthol, mebutanedione, isopentyl acetate; benzaldehyde; 3-phenylprop-2-enal; ethyl propionate; methyl anthranilate; limonene; ethyl 2-trans-4-cis-decadienoate, allyl hexanoate; ethyl maltol; ethylvanillin; methyl salicylate. The antimicrobial agent may be consisting of benzyl alcohol, chlorobutanol, methylparaben, propylparaben, phenol, ethylenediaminetetraacetic acid (EDTA), m-cresol, sorbic acid and its salts, benzoic acid and its salts, calcium propionate, sodium nitrite, sulfur dioxide, sodium bisulfite, and potassium hydrogen sulfite. A colorant may be selected from turmeric, cochineal, chlorophyllin, caramel, saffron, paprika, annatto, lycopene, betanin, pandanus amaryllifolius, clitoria ternatea or an artificial colorant of FD&C Blue No. 1, FD&C Blue No. 2, FD&C Green No. 3, FD&C Red No. 40, FD&C Red No. 3, FD&C Yellow No. 5, FD&C Yellow No. 6, Orange B, and Citrus Red 2.

### EXAMPLE 5

### Preparation of Oral Solution for Denture Adhesive Removal (E05)

An oral rinsing solution suitable for denture adhesive removal was prepared the same as in Example 1. A sample formulation is described in Table 7.

TABLE 7

| Ingredient | % |
|---|---|
| Polyethylene glycol 600 | 20 |
| Polyethylene glycol 300 | 20 |
| Xylitol | 5 |
| Benzyl alcohol | 2 |
| Peppermint | 0.5 |
| FD&C Green No. 3 | 0.1 |
| Purified Water | qs 100 |

Co-emulsification agents may be selected from glycerol, t-octylphenoxy-polyethoxyethanol, diethylene glycol monoethyl ether, propylene glycol, polyethylene glycols, methoxyl polyethylene glycol ethers, benzalkoniurn diloride, sodium lauryl sulfite, nonoxynol-9, octoxynol, cetylpyridinium chloride, and triglycerides. Sweetener may select from a sugar alcohol such mannitol or fucitol or sorbitol or xylitol. The suitable artificial flavor may be selected from peppermint, cherry, menthol, mebutanedione, isopentyl acetate; benzaldehyde; 3-phenylprop-2-enal; ethyl propionate; methyl anthranilate; limonene; ethyl 2-trans-4-cis-decadienoate, allyl hexanoate; ethyl maltol; ethylvanillin; methyl salicylate. The antimicrobial agent(s) may be consisting of benzyl alcohol, chlorobutanol, methylparaben, propylparaben, phenol, ethylenediaminetetraacetic acid (EDTA), m-cresol, sorbic acid and its salts, benzoic acid and its salts, calcium propionate, sodium nitrite, sulfur dioxide, sodium bisulfite, and potassium hydrogen sulfite. A colorant may be selected from turmeric, cochineal, chlorophyllin, caramel, saffron, paprika, annatto, lycopene, betanin, pandanus amaryllifolius, clitoria ternatea or an artificial colorant of FD&C Blue No. 1, FD&C Blue No. 2, FD&C Green No. 3, FD&C Red No. 40, FD&C Red No. 3, FD&C Yellow No. 5, FD&C Yellow No. 6, Orange B, and Citrus Red 2.

### EXAMPLE 6

### Comparison Study of Oral Solution for Denture Adhesive Removal

Two commercial denture adhesives were tested which is combined in an aqueous solution with different formulas. A variety of examples were tested, as well as a variety of formation temperatures. After a quick mixing by manual shaking or vortex, formulations were allowed to stand at room temperature. For comparison, the denture adhesive is only slightly emulsified (indicated with a negative sign) into water, while strong emulsifier (comparison made with the easiness to become homogenous a mixture and indicated with a triplet plus sign) may be used as a preferable remover. Therefore, for the purposes of this patent a denture adhesive remover is considered to have certain emulsifying power below about 35 degrees C. The results are shown in Table 8.

TABLE 8

| | PEG-Lipid | Emulsifiability¹ | | |
|---|---|---|---|---|
| Formula ID | Conc. (%) | 25° C. | 35° C. | 45° C. |

(a) Product A

| | | | | |
|---|---|---|---|---|
| E01 | 20 | ++ | +++ | +++ |
| E02 | 15 | ++ | +++ | +++ |
| E03 | 10 | ++ | ++ | +++ |
| E04 | 5 | + | ++ | ++ |
| E05 | 0 | + | + | ++ |
| Water | 0 | − | − | −/+ |

(b) Product B

| | | | | |
|---|---|---|---|---|
| E01 | 20 | ++ | +++ | +++ |
| E02 | 15 | ++ | +++ | +++ |
| E03 | 10 | ++ | ++ | +++ |
| E04 | 5 | + | ++ | ++ |
| E05 | 0 | + | + | ++ |
| Water | 0 | − | − | −/+ |

¹the ratio of adhesive to above aqueous solution was about 1 to 20 (w/v)
2 Score
(−) clear boundary/phase separation
(+) no boundary with some liquid phase
(++) no boundary and less liquid
(+++) form colloidal/gel

To achieve an optimal result for removing the denture adhesive residuals, it may be necessary rinsing multiple times. Additional techniques may be required following general procedures from using conventional mouthwash products. Swishing vigorously and thoroughly so that the rinse reaches the front and sides of intra-mouth equally, gargle by raising the chin while holding the rinse in the mouth. This is especially good for reaching the back of the tongue, where adhesive may accumulate. Continue rinsing for about 30 seconds, then completely spit the rinse from the mouth following by rinsing with water.

While preferred aspects and embodiments of the present invention have been described, those skilled in the art will recognize that other and further changes and modifications can be made without departing from the spirit of the invention, and all such changes and modifications should be understood to fall within the scope of the invention.

What claimed is:

1. A method for denture adhesive removal with aqueous oral hygiene solution, the aqueous oral hygiene solution being prepared by a method comprising steps (a) to (c), wherein (a) to (c) are:
a) adding at least one emulsification agent comprising amphophilic lipid conjugates of oleoyl-N-(3-aminopropyl)propane-1,3-diamine-monomethoxypolyethylene glycol ether lactobionate or oleoylpropanediamine-monomethoxypolyethylene glycol ether lactobionate or polyethylene glycol (PEG) based lipid or linear PEG-lipids containing alkyl chains of 8 to 20 carbons in an amount to provide a concentration in the aqueous oral hygiene solution at about 1% to about 30%, in a suitable container;
b) adding, to the container, a mixture of polyethylene glycol and propylene glycol as Co-emulsification agents in an amount to provide a concentration in the aqueous oral hygiene solution at about 1% to about 40%; and
c) mixing water into the container in an amount to provide the aqueous oral hygiene solution with a water content of at least 50%; and
the method for denture adhesive removal further comprising a step of rinsing the mouth with the prepared aqueous oral hygiene solution.

2. The method for denture adhesive removal of claim 1, wherein the at least one emulsification agent is selected from the group consisting of polyethylene glycol mono- or di-ester of fatty acids, polyoxyethylene mono- or di-ether of fatty alcohols, polyethoxylated castor oil, polyethylene glycol 1000 vitamin E, polysorbates, and polyoxyethylene ethers of a mixture of saturated fatty alcohols.

3. The method for denture adhesive removal of claim 1, wherein the Co-emulsification agent is selected from the group consisting of glycerol, diethylene glycol monoethyl ether, polyethylene glycols, methoxyl polyethylene glycol ethers, poloxamers, triglycerides, diglycerides, benzalkonium chloride, nonoxynol-9, octoxynol, and cetylpyridinium chloride.

4. The method for denture adhesive removal of claim 1 further comprising a step of adding a sweetener to the aqueous oral hygiene solution.

5. The method for denture adhesive removal of claim 4, wherein the sweetener is selected from the group consisting of mannitol, fucitol, sorbitol, and xylitol.

6. The method for denture adhesive removal of claim 1 further comprising a step of adding a flavor to the aqueous oral hygiene solution.

7. The method for denture adhesive removal of claim 6, wherein the flavor is selected from the group consisting of Peppermint (oil), Dare # 13174 (Cherry), Menthol (Mint), Butanedione (Buttery), Isopentyl acetate (Banana), Benzaldehyde (Bitter almond), 3-Phenylprop-2-enal (Cinnamon), Ethyl propionate (Fruity), Methyl anthranilate (Grape), Limonene (Orange), Ethyl 2-trans-4-cis-decadienoate (Pear), Allyl hexanoate (Pineapple), Ethyl maltol (Sugar, Cotton candy), Ethylvanillin (Vanilla), and Methyl salicylate (Wintergreen).

8. The method for denture adhesive removal of claim 1 further comprising a step of adding a colorant to the aqueous oral hygiene solution.

9. The method for denture adhesive removal of claim 8, wherein the colorant is selected from the group consisting of turmeric, cochineal, chlorophyllin, caramel, saffron, paprika, annatto, lycopene, betanin, pandanus amaryllifolius, clitoria ternatea or an artificial colorant of FD&C Blue No. 1, FD&C Blue No. 2, FD&C Green No. 3, FD&C Red No. 40, FD&C Red No. 3, FD&C Yellow No. 5, FD&C Yellow No. 6, Orange B, and Citrus Red 2.

10. The method for denture adhesive removal of claim 1 further comprising a step of adding an antimicrobial agent to the aqueous oral hygiene solution.

11. The method for denture adhesive removal of claim 10, wherein the antimicrobial agent is selected from the group consisting of benzyl alcohol, chlorobutanol, methylparaben, propylparaben, phenol, ethylenediaminetetraacetic acid, and m-cresol, sorbic acid and its salts, benzoic acid and its salts, calcium propionate, sodium nitrite, sulfur dioxide, sodium bisulfite, and potassium hydrogen sulfite.

12. The method for denture adhesive removal of claim 1, wherein a combination of the Co-emulsification agents and the at least one emulsification agent are added at a concentration providing between about 10 percent (w/v) to about 50 percent (w/v) of the combination in the aqueous oral hygiene solution.

13. The method for denture adhesive removal of claim 4, wherein the step of adding a sweetener to the aqueous oral hygiene solution comprises adding an amount of the sweetener to the aqueous oral hygiene solution to provide a concentration of the sweetener between about 0.5 percent (w/v) to about 10 percent (w/v).

14. The method for denture adhesive removal of claim 6, wherein the step of adding a flavor to the aqueous oral hygiene solution comprises adding an amount of the flavor to provide a concentration of the flavor between about 0.1 percent (w/v) to about 5 percent (w/v).

15. The method for denture adhesive removal of claim 8, wherein the step of adding a colorant to the aqueous oral hygiene solution comprises adding an amount of the colorant to provide a concentration of the colorant between about 0.1 percent (w/v) to about 2 percent (w/v).

16. The method for denture adhesive removal of claim 6, wherein the step of adding an antimicrobial agent to the aqueous oral hygiene solution comprises adding an amount to provide a concentration of the antimicrobial agent between about 0.1 percent (w/v) to about 3 percent (w/v).

17. The method for denture adhesive removal of claim 1, wherein the step of mixing in water comprises mixing in water in an amount to provide the aqueous oral hygiene solution with a water content between about 50 percent (v/v) to about 75 percent (v/v).

18. The method for denture adhesive removal of claim 1, wherein the aqueous oral hygiene solution has a pH between 3 and 7.

19. The method for denture adhesive removal of claim 17, further comprising a step of adding a buffer to the aqueous oral hygiene solution.

20. An oral composition for denture adhesive removal, comprising (a) to (c), wherein (a) to (c) are:
a) at least one emulsification agent comprising amphophilic lipid conjugates of oleoyl-N-(3-aminopropyl)propane-1,3-diamine-monomethoxypolyethylene glycol ether lactobionate or oleoylpropanediamine-monomethoxypolyethylene glycol ether lactobionate or polyethylene glycol (PEG) based lipid or linear PEG-lipids containing alkyl chains of 8 to 20 carbons at a concentration of about 1% to about 30%;
b) a mixture of polyethylene glycol and propylene glycol as Co-emulsification agents at a concentration of about 1% to about 40%; and
c) a water content of at least 50%.
